(19) United States
(12) Patent Application Publication
(10) Pub. No.: US 2005/0154432 A1
(43) Pub. Date: Jul. 14, 2005

(54) Apparatus for electromagnetic treatment of biological tissue

(76) Inventors: Vladimir Alexeevich Borisov, Moscow (RU); Alexei Vladimirovich Lukovkin, Moscow (RU); Nikolay Grigorievich Tkach, Moscow (RU)

Correspondence Address: LADAS & PARRY, 26 WEST 61ST STREET, NEW YORK, NY, 10023, US

(21) Appl. No.: 10/968,740

(22) Filed: Oct. 19, 2004

(30) Foreign Application Priority Data
Oct. 30, 2003 (RU) 2003-131643

(51) Int. Cl. A61F 2/00

(52) U.S. Cl. 607/101

(57) ABSTRACT

An apparatus for electromagnetic treatment of biological tissue comprises a housing, sources of pulsed radiation provided with reflectors, a diaphragm, a light filter and a light conducting device. The latter is made of material having a prescribed heat conductivity, containing dopants which enable enhancing the radiation within a prescribed wavelength range. The light conducting device is made as a monoblock or as a hollow body filled with a dispersion of fine particles of said material having a prescribed heat conductivity, said dopants, and a liquid whose refractive index is equal to the refractive index of the fine particles. In the preferred embodiment the apparatus comprises two sources of pulsed radiation and a means ensuring their simultaneous or alternate functioning.

### FIELD OF THE ART

The present invention relates to medical engineering and more particularly to an apparatus for electromagnetic treatment of biological tissue. The apparatus may be used in medical practice for treating pigmentation and vascular diseases of the skin, in cosmetological practice for tightening skin collagen, hair depilation, etc.

### DESCRIPTION OF PRIOR ART

Known in the art are apparatus for electromagnetic treatment of biological tissue, particularly those disclosed in U.S. Pat. Nos. 5,683,380, 5,885,273, 6,280,438, 6,514,243, and also in published U.S. Application No. 20030069567. Essentially, in all the above-mentioned documents an apparatus is described, comprising a housing in which a source of pulsed electromagnetic radiation is disposed, provided with a reflector for directing the radiation toward the outlet of the housing, at least one band-pass optical filter that passes a part of the radiation spectrum in a wavelength range suitable for carrying out the treatment, a light conducting device mounted at the outlet of the housing, which receives the part of the radiation passed by the filter and outputs this part of the radiation to the surface of biological tissue to be treated.

It is known that in treating biological tissue, particularly in hair depilation, the spectral composition of the radiation by which the treatment is performed is of great importance. The spectral window of the epidermis (the outer layer of the skin) has a range of 650 to 1100 nm, and the major part of the radiation must come within this range. The radiation which does not come into this range does not affect the hair follicle, because it does not reach it, but only heats the skin. An excessively strong heating of the skin not only does not produce any positive effect, but may even damage the skin. In particular, it is known that exposure to the short-wave range of the spectrum (violet, ultraviolet), even in not very large doses causes skin burns and also has mutagenic properties.

When using the above-described known apparatus, for protecting the skin from burns, a heat-absorbing gel plate is placed between the lower base of the light conducting device and the surface to be treated. This leads to reducing the effective output energy of the radiation, complicates the treatment procedure and adds to the cost of the procedure and of the equipment employed.

Therefore, the main object of the invention is to provide an apparatus for the electromagnetic treatment of biological tissue, which would allow reducing harmful thermal effect and be sufficiently simple.

Another object of the invention is to provide an apparatus for electromagnetic treatment of biological tissue, which would allow reducing the harmful thermal effect with a simultaneous enhancement of the radiation energy within a preset wavelength range.

Still another object of the invention is to enhance the apparatus efficiency by reducing losses of thermal power.

It is also an object of the invention to broaden the functional possibilities of the apparatus for electromagnetic treatment of biological tissue.

Yet another object of the invention is to extend the service life of the apparatus.

### ESSENCE OF THE INVENTION

According to one embodiment, the invention consists in that an apparatus for electromagnetic treatment of biological tissue comprises a housing, a source of pulsed elec tromagnetic radiation, disposed in this housing, a reflector located near said source for directing the radiation of said source toward an outlet opening of the housing, at least one light filter arranged between the source of radiation and the outlet opening of the housing, capable of passing a part of the electromagnetic radiation in a prescribed wavelength range, a light conducting device mounted in the outlet opening of the housing, having an upper base capable of receiving the part of the radiation passed by said filter, and a lower base adapted to contact the surface of biological tissue to be treated and to output the radiation which has passed through said light conducting device to said surface; the light conducting device being made of material having a prescribed heat conductivity.

The material of said light conducting device may be heat absorbing.

In this case the light conducting device is made from a material selected from the group comprising dense flint, quartz, leucosapphire.

For cases of application, said material may be heat insulating.

The light conducting device is made from a material selected from the group comprising glass, plexiglass.

In another embodiment it is expedient that the material from which the light conducting device is made should additionally contain dopants which enable selective enhancing the radiation within a prescribed wavelength range.

It has been found that such dopants not only enable enhancing the radiation within a prescribed wavelength range, but at the same time provide for a reduction of the radiation level in an undesirable (harmful) radiation wavelength range.

Ruby or alexandrite may be used as such dopants.

In one more embodiment, the light conducting device is made hollow, said upper and lower bases thereof being made of a material transparent for said electromagnetic radiation, and the hollow interior of the device being filled with a suspension of fine particles of said material having a prescribed heat conductivity, of said dopants which enable selective enhancing of the radiation in a prescribed wavelength range, and of a liquid dispersion medium whose refractive index is equal to the refractive index of said fine-dispersed material.

For enhancing the efficiency of operation of the light conducting device, it is desirable that the external surfaces of its side walls should be provided with at least one coating reflecting the electromagnetic radiation.

In still another embodiment of the invention, the apparatus comprises at least one additional source of pulsed electromagnetic radiation, analogous to the first source, disposed in the housing and provided with its own reflector for directing its radiation toward the outlet of the housing.

In this case it is expedient that the apparatus should be provided with a means for ensuring simultaneous or alternate functioning of these sources.

In yet another embodiment of the invention, the apparatus for electromagnetic treatment of biological tissue comprises a housing, at least one source of pulsed electromagnetic radiation, disposed near the source, for directing its radiation toward the outlet opening of the housing, a light conducting device disposed in the outlet of the housing, said device having an upper base capable of receiving the radiation of the source, reflected by a reflector, a lower base adapted to contact the surface of biological tissue to be treated and to output the radiation that has passed through said light conducting device to said surface, the light conducting device being made of material having a prescribed heat conductivity and containing dopants which enable enhancing the radiation within a prescribed wavelength range.

### BRIEF DESCRIPTION OF THE DRAWINGS

The invention will further be explained by a description of particular but not limiting variants of embodying thereof and by the accompanying drawings, in which:

FIG. 1 shows diagrammatically an apparatus for treating biological tissue with electromagnetic radiation, according to the invention;

FIG. 2 shows a light conducting device made as a monoblock, according to the invention;

FIG. 3 shows a light conducting device made as a hollow body filled with a suspension, according to the invention;

FIG. 4 shows another variant of an apparatus for electromagnetic treatment, according to the invention;

FIG. 5 shows spectral curves of electromagnetic radiation, where the wavelength in nm is plotted along the X-axis and the relative energy in percent is plotted along the Y-axis.

### DETAILED DESCRIPTION OF PREFERRED EMBODIMENTS OF THE INVENTION

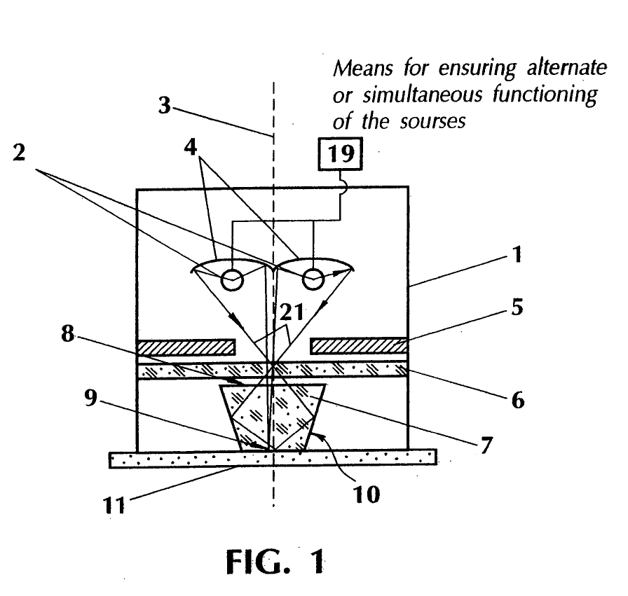

The apparatus for treating biological tissue with electromagnetic radiation, shown in FIG. 1 comprises a housing 1 open in the lower portion thereof, where an outlet is formed, and in the upper portion of which there is disposed at least one, in the example being described there are disposed two sources 2 of pulsed electromagnetic radiation, in the present case these sources being flashlamps. The sources 2 are disposed symmetrically with respect to the axis of symmetry 3 of the housing 1 and each source 2 is provided with its own reflector 4 which focuses the radiation of the source on the axis of symmetry 3 and directs the radiation toward the outlet of the housing 1. The reflectors 4 are made in the form of concave quadric surfaces. Besides, in the housing 1 behind the sources 2, as viewed along the radiation path, there are mounted a diaphragm 5, a light filter 6 and a light conducting device 7.

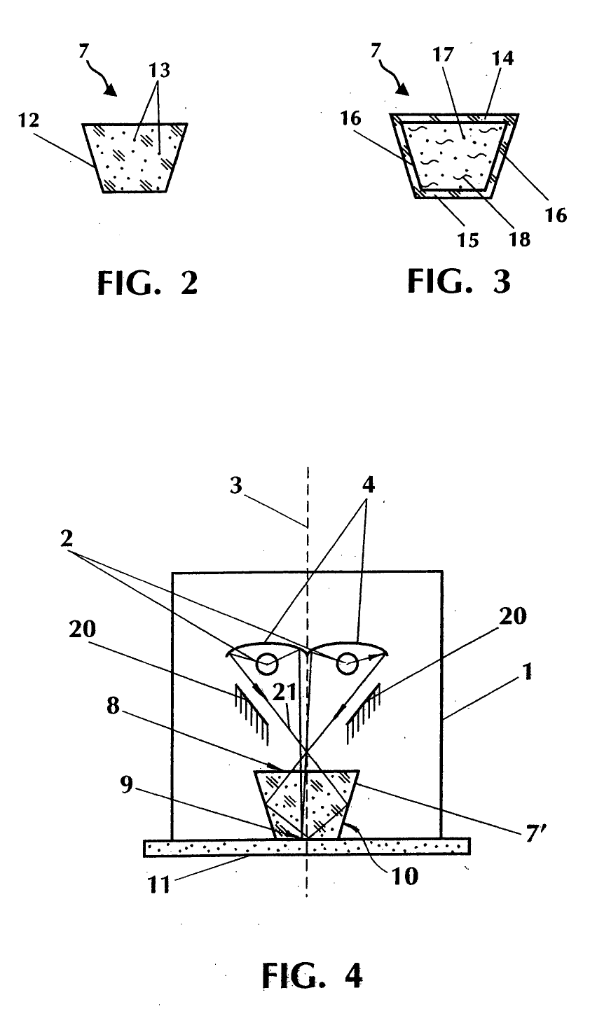

In the described exemplary embodiment the light conducting device is made as a prism having parallel upper and lower bases 8 and 9, respectively, and downwardly approaching inclined sides 10. The upper base 8 having a larger area is adapted to receive a flux of radiation coming from the source 2 of pulsed radiation, and the lower base 9 having a smaller area is adapted to contact directly the surface of the portion of biological tissue 11 to be treated. In one of the variants presented in FIG. 2 the prism is made as a monoblock 12 from a material transparent for the radiation and having a prescribed heat conductivity. The heat conductivity value is selected depending on the purpose of the device. For some applications, for instance, for the depilation of hair, when it is necessary to remove heat, this material has the heat conductivity which imparts thermo-conducting properties to it; for example, this can be dense flint, quartz or leucosapphire.

For other applications, for instance, for the therapeutic treatment of skin when treating dermatological diseases (acne etc.) and when thermal insulation of the locus of treatment is required, this material has a heat conductivity which provides thermo-insulating properties to it. In the present case glass or plexiglass can be used as such material.

The material of the prism further comprises dopants 13 which enable enhancing the radiation in a prescribed wavelength range. Fluorescent materials, ruby or alexandrite, can be selected as such dopants.

In the preferred embodiment of the light conducting device 7, shown in FIG. 3, this device comprises a hollow body whose upper and lower bases 14 and 15 are made of a material transparent for the electromagnetic radiation, and the internal surfaces of side walls 16 are made so as to provide total internal reflection of the radiation. The hollow interior of the body is filled with a suspension of fine (powder-like) particles 17 of material having a prescribed heat conductivity, this material containing dopants which enable enhancing the radiation in a prescribe wavelength range, and of a liquid dispersion medium 18 whose refractive index is equal to the refractive index of the fine-dispersed material. The external surfaces of the side walls 16 are also covered with at least one layer of a coating reflecting the electromagnetic radiation.

The apparatus is further provided with a power supply unit for the sources 2 of electromagnetic radiation (the power supply unit is not shown in the drawings), and with a means 19 (FIG. 1) for ensuring alternate or simultaneous functioning of the sources.

The variant of the apparatus shown in FIG. 4, unlike the variant of the apparatus shown in FIG. 1, comprises a light conducting device 71 which, owing to the selection of dopants which enable enhancing the radiation within a desired wavelength range, additionally performs the function of a light filter. Besides, two reflecting surfaces 20 which make it possible to reduce scattering of the radiation are arranged between the sources 2 of radiation and the upper base 8 of the light conducting device 7′ symmetrically with respect to the vertical optical axis of the device.

The apparatus of the invention operates as follows.

The electromagnetic treatment of biological tissue is effected with the aide of the pulsed sources 2 of light. The reflectors 4 direct their radiation 21 through an aperture of the diaphragm 5 and the light filter 6 to the lager base 8 of the light conducting device 7. The radiation inside the light conducting device is reflected from its side surfaces and comes to the smaller base 9, through which the radiation is outputted to the surface of the portion of biological tissue 10 to be treated, which is located under the base 9.

As the radiation passes through the light conducting device 7, due to the fact that this device is made of a material characterized by high heat conductivity and heat capacity values, for example, from leucosapphire or quartz doped with ruby, excess heat is absorbed by this material, and thereby the skin is protected from overheating, and at the same time the radiation energy in the prescribed wavelength range is enhanced. The use of the light conducting device according to the invention makes it possible to obviate the otherwise obligatory coating the skin surface with an interlayer of a thermo-absorbing gel.

The presence of dopants in the material of the light conducting device, which enable enhancing the radiation in a prescribed wavelength range, makes it possible to essentially improve the spectral characteristics of the apparatus as a whole owing to the enhancement of the output energy in the desired wavelength range, with a simultaneous reduction of energy in the undesirable harmful wavelength range of the radiation spectrum. The latter circumstance allows combining in certain cases the functions of the light conducting device and the light filter in one element and obviating the mounting of a separate light filter.

In particular, using an optical element containing dopants with fluorescence in the red and infrared regions makes it possible to enhance the output energy in the skin window region, for instance, when using alexandrite or ruby, by 7 to 15% with simultaneous reduction of the parasitic radiation energy in the ultraviolet and blue spectral regions down to 30%.

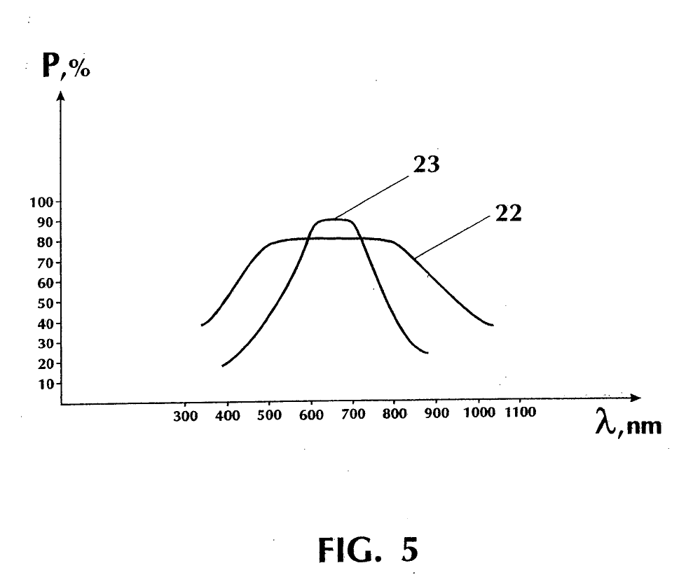

FIG. 5 shows spectral curves 22 and 23 of electromagnetic radiation, where curve 22 is the spectral characteristic of the radiation of source, and curve 23 is the spectral characteristic of the radiation at the outlet of the light conducting device 7, according to the invention. From a comparison of the curves it is seen that the radiation power at the outlet of the device 7 in the red wavelength region (600 to 700 nm) is approximately 10% higher than the power of the radiation source, while the power of radiation in the blue and far infrared regions of the spectrum at the outlet of the device 7 is 30 to 40% lower compared with the radiation of the source.

The apparatus may be successfully used with one source 2 of pulsed radiation. However, the use of two or more sources makes it possible to lower the employed discharge voltage of the lamp and to increase essentially its service life. In particular, the carried out experiments have shown that lowering the discharge voltage by 25 to 30% of the rated value made it possible to increase the lamp service life from 25,000 to 300,000 flashes.

Besides, lowering the discharge voltage makes it possible to increase the efficiency of the apparatus in the preferable red region of the spectrum. Thus, in the experiments, lowering the discharge voltage by 30% owing to simultaneous switching-on of two pulsed sources provided an increase in the output energy by 10 to 14% in the range of 650 to 670 nm and a decrease in the energy by 30 to 40% in the range of ultraviolet and blue wavelengths.

With alternate switching-on of the radiation sources, it is possible to organize successive flashes of each of the lamps in such a manner that there takes place practically continuous treatment of the skin with radiation. Usually the duration of the lamp flash may reach 100 msec, the duration of the lamp charging time is approximately the same. Continuous mode is of importance when carrying out some medical and cosmetological procedures. For instance, when treating pigmentous diseases of the skin, vascular diseases (angioma, telangiectasis), tightening skin collagen, depilation of thick light hair, continuous treatment for 200 to 500 msec is preferable.

In the exemplary embodiments radiation sources 2 are shown, arranged symmetrically with respect to the vertical axis 3 of the apparatus, but these sources may also be arranged one under the other along the axis 3 of the apparatus. The sources 2 may be provided with one common reflector. The radiation sources can be can be arranged asymmetrically, for example in case the sources have different power or different radiation spectrum.

1. An apparatus for electromagnetic treatment of biological tissue, comprising:
a housing;
a source of pulsed electromagnetic radiation, disposed in said housing;
said housing having an outlet for the output of the radiation of said source;
a reflector located near said source for directing the radiation of said source toward said outlet;
at least one light filter arranged between said source of radiation and said outlet, capable of passing a part of said electromagnetic radiation in a prescribed wavelength range;
a light conducting device mounted in said outlet of the housing, having an upper base capable of receiving the part of the radiation passed by said filter, and a lower base adapted to contact the surface of the biological tissue to be treated and to output the radiation which has passed through said light conducting device to said surface;
said light conducting device being made of material having a prescribed heat conductivity.

2. The apparatus as claimed in claim 1, wherein said material is heat absorbing.

3. The apparatus as claimed in claim 2, wherein said heat absorbing material is selected from the group comprising dense flint, quartz, leucosapphire.

4. The apparatus as claimed in claim 1, wherein said material is heat insulating.

5. The apparatus as claimed in claim 4, wherein said heat insulating material is selected from the group comprising glass, plexiglass.

6. The apparatus as claimed in claim 1, wherein said light conducting device is made of a material which further comprises dopants which enable selective enhancing of the radiation in a prescribed wavelength range.

7. The apparatus as claimed in claim 6, wherein said dopants are selected from the group comprising ruby and alexandrite.

8. The apparatus as claimed in claim 7, wherein said light conducting device is made hollow, said upper and lower bases thereof being made of a material transparent for said electromagnetic radiation, and the hollow interior thereof being filled with a suspension of fine particles of said material having a prescribed heat conductivity, of said dopants which enable selective enhancing of the radiation in a prescribed wavelength range, and of a liquid dispersion medium whose refractive index is equal to the refractive index of said fine-dispersed material.

9. The apparatus as claimed in claim 1, wherein the external surfaces of the side walls of said light conducting device are provided with at least one coating reflecting said electromagnetic radiation.

10. The apparatus as claimed in claim 1, which comprises at least one additional source of pulsed electromagnetic radiation, analogous to the first said source, disposed in said housing and provided with its own reflector for directing the radiation of said additional source toward said outlet of the housing.

11. The apparatus as claimed in claim 10, wherein said sources of pulsed radiation are provided with a means ensuring their joint functioning.

12. The apparatus as claimed in claim 10, wherein said sources of pulsed radiation are provided with a means ensuring their alternate functioning.

13. An apparatus for electromagnetic treatment of biological tissue, comprising:
a housing;
a source of pulsed electromagnetic radiation, disposed in said housing;
said housing having an outlet for the output of the radiation of said source;
a reflector located near said source for directing the radiation of said source toward said outlet;
a light conducting device mounted in said outlet of the housing, having an upper base capable of receiving the radiation of said source, reflected by said reflector, a lower base adapted to contact the surface of the biological tissue to be treated and to output the radiation which has passed through said light conducting device to said surface;
said light conducting device being made of material having a prescribed heat conductivity, containing dopants which enable enhancing said radiation within a prescribed wavelengthrange.
